(19) United States
(12) Patent Application Publication
(10) Pub. No.: US 2020/0061163 A1
(43) Pub. Date: Feb. 27, 2020

(54) METHODS OF TREATING CANCER WITH THE COMBINATION OF GRANZYME A GENE DELIVERY AND HSP90 INHIBITION

(71) Applicant: ARIZONA BOARD OF REGENTS ON BEHALF OF ARIZONA STATE UNIVERSITY, SCOTTSDALE, AZ (US)

(72) Inventors: Kaushal REGE, Gilbert, AZ (US); Rajeshwar NITIYANANDAN, Tempe, AZ (US)

(73) Assignee: ARIZONA BOARD OF REGENTS ON BEHALF OF ARIZONA STATE UNIVERSITY, SCOTTSDALE, AZ (US)

(21) Appl. No.: 16/545,894

(22) Filed: Aug. 20, 2019

Related U.S. Application Data
(60) Provisional application No. 62/720,484, filed on Aug. 21, 2018.

(51) Int. Cl. A61K 38/48; A61K 31/395; A61K 31/404; A61K 31/201; A61P 35/00

(52) U.S. Cl. CPC A61K 38/482; A61K 31/395; A61K 31/404; A61K 48/00; A61P 35/00; C12Y 304/21078; A61K 31/201

(57) ABSTRACT

A method for inhibiting and/or treating cancer in a subject in need thereof is disclosed. The method can include administering to a subject an effective amount of an HSP90 inhibitor; and administering to the subject an effective amount of Granzyme A, thereby treating and/or inhibiting the cancer in the subject in need thereof. A composition including an effective amount of an HSP90 inhibitor and an effective amount of Granzyme A is also disclosed.

### CROSS-REFERENCE TO RELATED APPLICATION

This application claims the benefit of U.S. Patent Application No. 62/720,484, filed on Aug. 21, 2018, which is hereby incorporated by reference in its entirety.

### FIELD

This disclosure relates to cancer and in particular, the treatment of cancer by the combination of Granzyme A expression and HSP90 inhibition.

### BACKGROUND

Bladder and ovarian cancer account for nearly 200,000 new cases each year in the United States alone and resulting in deaths of nearly 50,000 people. Current treatments used to treat various types of cancer tend to work by poisoning or killing the cancerous cell. Unfortunately, treatments that are toxic to cancer cells typically tend to be toxic to healthy cells as well. Moreover, the heterogeneous nature of tumors is one of the primary reasons that effective treatments for cancer remain elusive. Current mainstream therapies such as chemotherapy and radiotherapy tend to be used within a narrow therapeutic window of toxicity. These types of therapies are considered blunt tools that have limited applicability due to the varying types of tumor cells and the limited window in which these treatments can be administered. Recurrence rate after conventional therapy is high indicating a need for a better class of treatments for these cancers.

### SUMMARY

Disclosed herein are methods for inhibiting and/or treating cancer in a subject in need thereof. In some embodiments, the method includes administering to a subject an effective amount of an HSP90 inhibitor and administering to the subject an effective amount of Granzyme A, thereby treating and/or inhibiting the cancer in the subject in need thereof. Also, disclosed are compositions including an effective amount of an HSP90 inhibitor and an effective amount of Granzyme A.

The foregoing and other features of the disclosure will become more apparent from the following detailed description, which proceeds with reference to the accompanying figures.

### BRIEF DESCRIPTION OF THE DRAWINGS

FIG. 1 is a bar graph showing cancer cell ablation efficacy following delivery of pGranzyme A using different polymer: pDNA complexes at a weight ratio of 25:1 in UMUC3 cells. Data are presented as mean±one standard deviation of three independent experiments (n=3). * indicates p-value <0.05 when comparing polymer complexed with pGranzyme to polymers complexed with pGFP. Statistical analysis was done using Student's T-Test.

FIG. 2 is a bar graph showing cancer cell ablation efficacy following delivery of pGranzyme A using different polymer: pDNA complexes at a weight ratio of 25:1 in the presence of PARP inhibitor (Olaparib) in UMUC3 cells. Data are presented as mean±one standard deviation of three independent experiments (n=3). * indicates p-value <0.05 when comparing treatment with polymer complexed with pGranzyme in presence and absence of PARP inhibitor (Olaparib). Statistical analysis was done using student's t-Test.

FIG. 3 is a bar graph showing bladder cancer cell ablation efficacy of Granzyme A after pre-treatment with HSP90 inhibitor 17-AAG. Transgene expression studies were carried out with PGC18 polymer:pDNA complexes (25:1) in UMUC3 cells. Data are presented as mean±one standard deviation of three independent experiments (n=3). * indicates p-value <0.05 comparing treatment with Granzyme A polyplexes in presence of 17-AAG to pGranzyme polyplex only treatment. Statistical analysis using student's t-Test.

FIG. 4 is a bar graph showing the efficacy of HSP90 siRNA in increasing Granzyme A mediated cytotoxicity. siRNA delivery performed using PRC6 polymer. pGranzyme transfection performed using PGC18 polymer:pDNA compexes (25:1) in UMUC3 cells. Data is presented as mean±one standard deviation of three independent experiments (n=3). * indicates p-value <0.05 comparing transfection with Granzyme A polyplexes post siRNA treatment to pGranzyme polyplex only treatment.

FIG. 5 is a bar graph showing the generation of ROS in UMUC3 cells in different treatment conditions was measured after 36 h using transformation of 2′,7′-dichlorofluorescin diacetate (DCFDA) to the fluorescent 2′,7-dichlorofluorescein (DCF compound). Data are represented as the mean±one standard deviation of three independent experiments. Statistical Analysis was performed using one-way ANOVA.

FIG. 6 is a bar graph showing cancer cell ablation efficiency of pTRAIL in the presence of HSP90 inhibitor 17-AAG. Transfection performed using PGC18 Polymer:pTRAIL complexes (25:1) in UMUC3 cells. Data are presented as mean±one standard deviation of three independent experiments (n=3). * indicates p-value <0.05 comparing treatment with pTRAIL polyplexes in presence of 17-AAG to pTRAIL polyplex only treatment. Statistical Analysis performed using Student's t-Test.

FIG. 7 is a bar graph showing ovarian cancer cell ablation efficiency screen using HSP90 inhibitor and pGranzyme A combination treatment. Transgene expression studies were performed using PGC18 polymer:pDNA complexes (25:1 and 50:1) in A2780 ovarian cancer cells. Data are presented as mean±one standard deviation of three independent experiments (n=3). * indicates p-value <0.05 comparing treatment with pGranzyme A polyplexes in presence of 17-AAG to pGranzyme polyplex only treatment. Statistical analysis was performed using One-way ANOVA.

FIG. 8 is a digital image of Western blot analysis performed to determine APE-1 expression under different treatment conditions.

FIG. 9 is a bar graph showing 17-AAG and pGranzyme A combination treatment leads to reduction in levels of APE 1. Significant difference observed between cells treated only with 17-AAG and cells treated with 17-AAG and pGranzyme. Data are represented as mean±one standard deviation of three independent experiments. * indicates a p-value of less 0.05. Statistical analysis was performed using one way ANOVA.

FIG. 10 are two graphs showing the mitochondrial membrane potential was measured after 24 h (upper) and 48 hrs (lower) after staining treated cells using JC-1 dye.

FIG. 11 is a bar graph showing combination treatment with 17-AAG (HSP90 inhibitor) and CRT 0044876 (APE-1 Inhibitor) showed a significant decrease in cell viability in both A2780 and UMUC3 cells respectively. Data are represented as mean±one standard deviation of three independent experiments. **** indicates a p-value of less 0.0001. Statistical analysis was performed using one way ANOVA followed post hoc Tukey's test.

FIG. 12 is a bar graph showing that combination treatment with 17-AAG (HSP90 inhibitor) and E3330 (APE-1 Redox Inhibitor) showed a significant decrease in cell viability in both A2780 and UMUC3 cells respectively. Data are represented as mean±one standard deviation of two independent experiments. **** indicates a p-value of less 0.0001, *** indicates a p-value of 0.001 and ** indicates a p-value of 0.01. Statistical analysis was performed using one way ANOVA followed post hoc Tukey's test.

FIG. 13 is a bar graph showing that combination treatment with 17-AAG HSP90 siRNA and CRT 0044876 (APE-1 Inhibitor). APE-1 inhibition 4 days post siRNA transfection showed a large increase in cell death in comparison to scrambled siRNA treatments. Data are represented as mean±one standard deviation of two independent experiments and ** indicates a p-value of 0.01. Statistical analysis was performed using one way ANOVA followed post hoc Tukey's test.

FIG. 14 is a bar graph showing that combination treatment with 17-AAG HSP90 siRNA and CRT 0044876 (APE-1 Inhibitor). APE-1 inhibition 4 days post siRNA transfection showed a large increase in cell death in comparison to scrambled siRNA treatments. Data are represented as mean±one standard deviation of two independent experiments and ** indicates a p-value of 0.01. Statistical analysis was performed using one way ANOVA followed post hoc Tukey's test.

FIG. 15 is a bar graph showing the generation of ROS in A2780 cells in different combination treatment conditions was measured after 36 h using transformation of 2′,7′-dichlorofluorescin diacetate (DCFDA) to the fluorescent 2′,7-dichlorofluorescein (DCF compound). Data are represented as the mean mean±one standard deviation of three independent experiments. Statistical Analysis was performed using one-way ANOVA.

FIG. 16 is an illustration depicting the Granzyme A and HSP90 inhibition induced cell death.

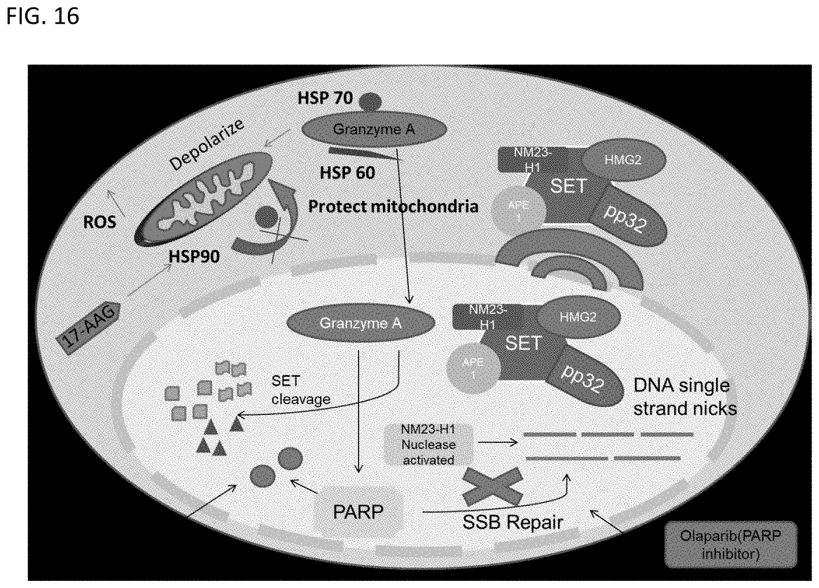

### DETAILED DESCRIPTION OF SEVERAL EMBODIMENTS

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCR Publishers, Inc., 1995 (ISBN 1-56081-569-8); and other similar references.

Unless otherwise explained, all technical and scientific terms used herein have the same meaning as commonly understood by one of ordinary skill in the art to which this disclosure belongs. Although methods and materials similar or equivalent to those described herein can be used in the practice or testing of this disclosure, suitable methods and materials are described below. In addition, the materials, methods, and examples are illustrative only and not intended to be limiting. All references presented herein are incorporated by references in their entirety.

The following explanations of terms and methods are provided to better describe the present compounds, compositions and methods, and to guide those of ordinary skill in the art in the practice of the present disclosure. It is also to be understood that the terminology used in the disclosure is for the purpose of describing particular embodiments and examples only and is not intended to be limiting.

As used herein, the singular forms “a,” “an,” and “the” are intended to include the plural forms as well, unless the context clearly indicates otherwise.

As used herein, the term “and/or” refers to and encompasses any and all possible combinations of one or more of the associated listed items, as well as the lack of combinations when interpreted in the alternative (“or”).

As used herein, “one or more” or at least one can mean one, two, three, four, five, six, seven, eight, nine, ten or more, up to any number.

As used herein, the term “comprises” means “includes.” Hence “comprising A or B” means including A, B, or A and B.

An “effective amount” or “therapeutically effective amount” refers to an amount of a compound or composition of this invention that is sufficient to produce a desired effect, which can be a therapeutic and/or beneficial effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an effective amount or therapeutically effective amount in any individual case can be determined by one of ordinary skill in the art by reference to the pertinent texts and literature and/or by using routine experimentation. (See, for example, Remington, The Science and Practice of Pharmacy (latest edition)).

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, rodents (e.g., mice, rats, etc.) and the like. Preferably, the subject is a human patient. In particular embodiments, the subject of this disclosure is a human subject.

The term “a cell” as used herein includes a single cell as well as a plurality or population of cells. Administering an agent to a cell includes both in vitro and in vivo administrations. The composition disclosed herein van be administered to a cell.

A “subject in need thereof” or “a subject in need of” is a subject known to have, or is suspected of having a cancer.

As used herein, the term “cancer” refers to a malignant neoplasm that has undergone characteristic anaplasia with loss of differentiation, increased rate of growth, invasion of surrounding tissue, and is capable of metastasis.

Residual cancer is cancer that remains in a subject after any form of treatment given to the subject to reduce or eradicate cancer. Metastatic cancer is a cancer at one or more sites in the body other than the site of origin of the original (primary) cancer from which the metastatic cancer is derived. Local recurrence is reoccurrence of the cancer at or near the same site (such as in the same tissue) as the original cancer.

As used herein, the term “chemotherapeutic agent” refers to any chemical agent with therapeutic usefulness in the treatment of diseases characterized by abnormal cell growth. Such diseases include tumors, neoplasms, and cancer, as well as diseases characterized by hyperplastic growth such as psoriasis. In one embodiment, a chemotherapeutic agent is an agent of use in treating cancer, such as an anti-neoplastic agent. In one embodiment, a chemotherapeutic agent is a radioactive compound. One of skill in the art can readily identify a chemotherapeutic agent of use (see for example, Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer and Berkery. (eds): Oncology Pocket Guide to Chemotherapy, 2nd ed. St. Louis, Mosby-Year Book, 1995; Fischer Knobf, and Durivage (eds): The Cancer Chemotherapy Handbook, 4th ed. St. Louis, Mosby-Year Book, 1993. Combination chemotherapy is the administration of more than one agent to treat cancer.

As used herein, the term “inhibiting or treating a disease” refers to inhibiting the full development of a disease or condition, for example, for example cancer. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, such a metastasis, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology, for example metastatic cancer.

As used herein, the term “Heat Shock Protein 90 (HSP90) inhibitor” refers to a substance that inhibits that activity of the Hsp90 heat shock protein. Hsp90 inhibitors include the natural products geldanamycin and radicicol as well as semisynthetic derivatives 17-N-Allylamino-17-demethoxygeldanamycin (17AAG).

As used herein, the terms “pharmaceutical” and “therapeutic agent” refer to a chemical compound or a composition capable of inducing a desired therapeutic or prophylactic effect when properly administered to a subject or a cell.

As used herein, the term “isolated” refers to a biological component that has been substantially separated or purified away from other biological components in which the component naturally occurs, such as other chromosomal and extrachromosomal DNA, RNA, and proteins. Proteins, peptides and nucleic acids that have been “isolated” include proteins purified by standard purification methods. The term also embraces proteins or peptides prepared by recombinant expression in a host cell as well as chemically synthesized proteins, peptides and nucleic acid molecules.

Isolated does not require absolute purity, and can include protein, peptide, or nucleic acid molecules that are at least 50% isolated, such as at least 75%, 80%, 90%, 95%, 98%, 99%, or even 99.9% isolated.

As used herein, the term “nucleic acid” refers to a polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer.

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (for example, rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Recombinant nucleic acid” refers to a nucleic acid having nucleotide sequences that are not naturally joined together. This includes nucleic acid vectors comprising an amplified or assembled nucleic acid which can be used to transform a suitable host cell. A host cell that comprises the recombinant nucleic acid is referred to as a “recombinant host cell.” The gene is then expressed in the recombinant host cell to produce, such as a “recombinant polypeptide.” A recombinant nucleic acid may serve a non-coding function (such as a promoter, origin of replication, ribosome-binding site, etc.) as well.

As used herein, “operably linked” refers to when first nucleic acid sequence is linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

As used herein, a “pharmaceutical agent or drug” refers to a chemical compound or composition capable of inducing a desired therapeutic or prophylactic effect when properly administered to a subject. Pharmaceutically acceptable useful in this disclosure are conventional. Remington's Pharmaceutical Sciences, by E. W. Martin, Mack Publishing Co., Easton, Pa., 19th Edition (1995), describes compositions and formulations suitable for pharmaceutical delivery of the proteins and other compositions herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions, powder, pill, tablet, or capsule forms, conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

As used herein, the terms “tumor”, “tumor cells”, “cancer” and “cancer cells”, (used interchangeably) refer to cells that exhibit abnormal growth, characterized by a significant loss of control of cell proliferation or cells that have been immortalized. The term “cancer” or “tumor” includes metastatic as well as non-metastatic cancer or tumors. As used herein, “neoplastic” or “neoplasm” broadly refers to a cell or cells that proliferate without normal growth inhibition mechanisms, and therefore includes benign tumors, in addition to cancer as well as dysplastic or hyperplastic cells. A tumor that invades the surrounding tissue and/or can metastasize is referred to as “malignant.” Neoplasia is one example of a proliferative disorder.

As used herein, the term “transformation” encompasses all techniques by which a nucleic acid molecule might be introduced into such a cell, including transfection with viral vectors, transformation with plasmid vectors, and introduction of DNA by electroporation, lipofection, and particle gun acceleration. A “transformed cell” is a cell into which has been introduced a nucleic acid molecule by molecular biology techniques.

As used herein, the terms “tumor”, “tumor cells”, “cancer” and “cancer cells”, (used interchangeably) refer to cells that exhibit abnormal growth, characterized by a significant loss of control of cell proliferation or cells that have been immortalized. The term “cancer” or “tumor” includes metastatic as well as non-metastatic cancer or tumors. As used herein, “neoplastic” or “neoplasm” broadly refers to a cell or cells that proliferate without normal growth inhibition mechanisms, and therefore includes benign tumors, in addition to cancer as well as dysplastic or hyperplastic cells.

As used herein, the term “vector” is a nucleic acid molecule as introduced into a host cell, thereby producing a transformed host cell. Recombinant DNA vectors are vectors having recombinant DNA. A vector can include nucleic acid sequences that permit it to replicate in a host cell, such as an origin of replication. A vector can also include one or more selectable marker genes and other genetic elements known in the art. Viral vectors are recombinant DNA vectors having at least some nucleic acid sequences derived from one or more viruses.

### Description of Several Embodiments

Immune cells in humans, for example, cytotoxic T-Cells and natural killer (NK) cells release a class of enzymes named granzymes which induce programmed cell death in cells which have either become infected or cancerous. Granzymes are serine proteases and there are 5 different types of granzymes present in humans; of which Granzyme A is the most abundant and uses a caspase independent pathway to induce cell death. Granzyme A mediates cell death through depolarization of mitochondria. APE1 is a DNA repair and redox factor enzyme which is produced in response to DNA damage or upregulation of Reactive oxygen species (ROS) in cells. The purpose of this enzyme is to prevent cell death due to ROS mediated DNA damage. Heat shock protein 90 or HSP90 is a molecular chaperone protein which also has mitochondrial protective properties. Through the inhibition of HSP90 one is able to remove this mitochondrial protective property while also inducing upregulation of ROS inside cells.

One of the problems associated with both heat shock protein inhibitors and APE1 inhibitors is the need for high dosages to get efficient killing of cancer cells. Unfortunately, at these high dosages, there is significant toxicity to the normal tissues in patients. This is especially true for heat shock protein inhibitors which have been shown to be toxic in clinical trials.

To overcome these and other problems, the inventors have developed a combination therapy avoids the pitfalls associated with high concentrations of these drugs. The inventors have shown that they can achieve a similar or better cancer killing effect when combined in low doses with each other. As disclosed herein, the inventors have demonstrated that HSP90 inhibitor in combination Granzyme A plasmid delivery shows synergistic ablation caused by this combination treatment in-vitro in cancer cells. The inventors investigated delivery of a plasmid DNA (pEF-Granzyme A) that expresses the Granzyme A protein using aminoglycoside derived polymers to induce death in cancer cells in-vitro. When combined with the delivery of Granzyme A plasmid, this results in improved efficacy of the produced Granzyme A protein as it is able to depolarize the mitochondria effectively and results in subsequent DNA damage and cancer cell ablation. Similarly, inhibition of APE1 protein which works as both a DNA repair protein and ROS scavenger, removes the repair mechanism/ROS scavenging.

Aspects of this disclosure concern a method of treating and/or inhibiting cancer, such as cancer cells, for example in a subject. In certain embodiments, the method includes administering to a subject an effective amount of an HSP90 (Heat shock protein 90) inhibitor, and administering to the subject Granzyme A plasmid and/or inhibitors of APE1/Ref1 (DNA repair and Redox factor protein). In embodiments, a method for inhibiting and/or treating cancer in a subject in need thereof includes administering to a subject an effective amount of an HSP90 inhibitor and administering to the subject an effective amount of Granzyme A, thereby treating and/or inhibiting the cancer in the subject in need thereof. In embodiments, methods include selecting a subject with cancer or suspected of having cancer. “Treating” a disease state refers to an approach for obtaining beneficial or desired results, including clinical results. Beneficial or desired clinical results can include, but are not limited to, alleviation or amelioration of one or more symptoms or conditions, diminishment of extent of disease, stabilization of the state of disease, prevention of development of disease, prevention of spread of disease, delay or slowing of disease progression, delay or slowing of disease onset, amelioration or palliation of the disease state, and remission (whether partial or total), whether detectable or undetectable. “Treating” can also mean prolonging survival of a patient beyond that expected in the absence of treatment. “Treating” can also mean inhibiting the progression of disease, slowing the progression of disease temporarily, although more preferably, it involves halting the progression of the disease permanently. As will be understood by a skilled person, results may not be beneficial or desirable if, while improving a specific disease state, the treatment results in adverse effects on the patient treated that outweigh any benefits effected by the treatment.

A cancer may be diagnosed using criteria generally accepted in the art, including the presence of a malignant tumor.

Cancers include malignant tumors that are characterized by abnormal or uncontrolled cell growth. Subjects treated using the methods provided herein may have cancer, or have had a cancer treated in the past (e.g., treated with surgical resection, chemotherapy, radiation therapy). Exemplary cancers that can benefit from the methods, compositions, and kits provided herein include cancers of the heart (e.g., sarcoma (angiosarcoma, fibrosarcoma, rhabdomyosarcoma, liposarcoma), myxoma, rhabdomyoma, fibroma, lipoma and teratoma), lung (e.g., bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma); gastrointestinal tract (e.g., esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma), genitourinary tract (e.g., kidney (adenocarcinoma, Wilm's tumor, nephroblastoma, lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma), liver (e.g., hepatoma (hepatocellular carcinoma), cholangiocarcinoma, hepatoblastom, angiosarcoma, hepatocellular adenoma, hemangioma), bone (e.g., osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor, chordoma, osteochronfroma (osteocartilaginous exostoses), benign chondroma, chondroblastoma, chondromyxofibroma, osteoid osteoma and giant cell tumors), nervous system (e.g., skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiforme, oligodendroglioma, schwannoma, retinoblastoma, congenital tumors), spinal cord (neurofibroma, meningioma, glioma, sarcoma)), gynecological cancers (e.g., uterus (endometrial carcinoma), cervix (cervical carcinoma, pre-tumor cervical dysplasia), ovaries (ovarian carcinoma, serous cystadenocarcinoma, mucinous cystadenocarcinoma, endometrioid tumors, celioblastoma, clear cell carcinoma, unclassified carcinoma, granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma, embryonal rhabdomyosarcoma, fallopian tubes (carcinoma)), hematologic cancers (e.g., blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma)), skin (e.g., malignant melanoma, basal cell carcinoma, squamous cell carcinoma, Karposi's sarcoma, moles, dysplastic nevi, lipoma, angioma, dermatofibroma, keloids, psoriasis), and adrenal glands (e.g., neuroblastoma). In a specific example, the cancer is one or more of leukemia (such as acute myelogenous leukemia or chronic myelogenous leukemia), bladder cancer, breast cancer, stomach cancer, lung cancer, ovarian cancer, thyroid cancer, soft tissue sarcoma, multiple myeloma, Bacille Calmette-Guerin (BCG)-refractory carcinoma in situ, neuroblastoma, gastric cancer or lymphoma (such as Hodgkin's lymphoma).

Inhibitors that can be used in the methods, compositions, and kits provided herein include small molecule inhibitors, inhibitory RNAs (such as siRNA or shRNA) that inhibit the expression, an antibodies, or combinations thereof.

Methods of designing and making RNAi molecules are known in the art. Generally, the term “antisense” refers to a nucleic acid capable of hybridizing to a portion of a RNA sequence by virtue of some sequence complementarity. Antisense nucleic acids can be oligonucleotides that are double-stranded or single-stranded, RNA or DNA or a modification or derivative thereof, which can be directly administered or can be produced intracellularly by transcription of exogenous, introduced sequences. Antisense nucleic acids range from about 6 to about 100 nucleotides in length. Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage. Ribozyme molecules include one or more sequences and include the well-known catalytic sequence responsible for mRNA cleavage (see U.S. Pat. No. 5,093,246). A ribozyme gene directed against HSP90 can be delivered to a subject endogenously (where the ribozyme coding gene is transcribed intracellularly) or exogenously (where the ribozymes are introduced into a cell, for example by transfection). Methods describing endogenous and exogenous delivery are provided in Marschall et al. (Cell Mol. Neurobiol. 14:523-38, 1994). Short interfering or interrupting RNA (siRNA) is a double-stranded RNA that can induce sequence-specific post-transcriptional gene silencing, thereby decreasing or even inhibiting gene expression. In some examples, siRNA molecules are about 19-23 nucleotides in length, such as at least 21 nucleotides, for example at least 23 nucleotides. In embodiments, the HSP90 inhibitor is one or more of a small molecule inhibitor of HSP90 activity, an inhibitory RNA that inhibits the expression of HSP90 protein, or an antibody that specifically binds HSP90. In embodiments, the small molecule inhibitor of HSP90 activity is 17-AAG (Tanespimycin).

In embodiments, administering to the subject an effective amount of Granzyme A, comprises administering to the subject a nucleic acid encoding Granzyme A, wherein the nucleic acid encoding Granzyme A is operatively linked to a promoter. In embodiments, the nucleic acid encoding Granzyme A is operatively linked to a promoter comprises an expression vector. In embodiments, the expression vector comprises pEF-Granzyme A plasmid DNA. A nucleic acid encoding an Granzyme A can be cloned or amplified by in vitro methods, such as the polymerase chain reaction (PCR), the ligase chain reaction (LCR), the transcription-based amplification system (TAS), the self-sustained sequence replication system (3SR) and the QP replicase amplification system (QB). For example, a polynucleotide encoding the protein can be isolated by polymerase chain reaction of cDNA using primers based on the DNA sequence of the molecule. A wide variety of cloning and in vitro amplification methodologies are well known to persons skilled in the art. PCR methods are described in, for example, U.S. Pat. No. 4,683,195; Mullis et al., Cold Spring Harbor Symp. Quant. Biol. 51:263, 1987; and Erlich, ed., PCR Technology, (Stockton Press, N Y, 1989). Polynucleotides also can be isolated by screening genomic or cDNA libraries with probes selected from the sequences of the desired polynucleotide under stringent hybridization conditions.

The polynucleotides encoding Granzyme A include a recombinant DNA which is incorporated into a vector in an autonomously replicating plasmid or virus or into the genomic DNA of a prokaryote or eukaryote, or which exists as a separate molecule (such as a cDNA) independent of other sequences. The nucleotides can be ribonucleotides, deoxyribonucleotides, or modified forms of either nucleotide. The term includes viral vectors also prepared for encoding Granzyme A. A number of viral vectors have been constructed, including polyoma, SV40 (Madzak et al., 1992, J. Gen. Virol., 73: 15331536), adenovirus (Berkner, 1992, Cur. Top. Microbiol. Immunol., 158:39-6; Berliner et al., 1988, Bio Techniques, 6:616-629; Gorziglia et al., 1992, J. Virol., 66:4407-4412; Quantin et al., 1992, Proc. Nad. Acad. Sci. USA, 89:2581-2584; Rosenfeld et al., 1992, Cell, 68: 143-155; Wilkinson et al., 1992, Nucl. Acids Res., 20:2233-2239; Stratford-Perricaudet et al., 1990, Hum. Gene Ther., 1:241-256), vaccinia virus (Mackett et al., 1992, Biotechnology, 24:495-499), adeno-associated virus (Muzyczka, 1992, Curr. Top. Microbiol. Immunol., 158:91-123; On et al., 1990, Gene, 89:279-282), herpes viruses including HSV and EBV (Margolskee, 1992, Curr. Top. Microbiol. Immunol., 158:67-90; Johnson et al., 1992, J. Virol., 66:29522965; Fink et al., 1992, Hum. Gene Ther. 3: 11-19; Breakfield et al., 1987, Mol. Neurobiol., 1:337-371; Fresse et al., 1990, Biochem. Pharmacol., 40:2189-2199), Sindbis viruses (H. Herweijer et al., 1995, Human Gene Therapy 6: 1161-1167; U.S. Pat. Nos. 5,091,309 and 5,2217,879), alphaviruses (S. Schlesinger, 1993, Trends Biotechnol. 11: 18-22; I. Frolov et al., 1996, Proc. Natl. Acad. Sci. USA 93: 11371-11377) and retroviruses of avian (Brandyopadhyay et al., 1984, Mol. Cell Biol., 4:749-754; Petropouplos et al., 1992, J. Virol., 66:3391-3397), murine (Miller, 1992, Curr. Top. Microbiol. Immunol., 158: 1-24; Miller et al., 1985, Mol. Cell Biol., 5:431-437; Sorge et al., 1984, Mol. Cell Biol., 4: 1730-1737; Mann et al., 1985, J. Virol., 54:401-407), and human origin (Page et al., 1990, J. Virol., 64:5370-5276; Buchschalcher et al., 1992, J. Virol., 66:2731-2739). Baculovirus (Autographa californica multinuclear polyhedrosis virus; AcMNPV) vectors are also known in the art, and may be obtained from commercial sources (such as PharMingen, San Diego, Calif.; Protein Sciences Corp., Meriden, Conn.; Stratagene, La Jolla, Calif.).

Thus, in one embodiment, the polynucleotide encoding Granzyme A is included in a viral vector. Suitable vectors include retrovirus vectors, orthopox vectors, avipox vectors, fowlpox vectors, capripox vectors, suipox vectors, adenoviral vectors, herpes virus vectors, alpha virus vectors, baculovirus vectors, Sindbis virus vectors, vaccinia virus vectors, adeno-associated virus vectors, and polio virus vectors. Specific exemplary vectors are poxvirus vectors such as vaccinia virus, fowlpox virus and a highly attenuated vaccinia virus (MVA), adenovirus, baculovirus and the like.

Vectors that encode Granzyme A generally include at least one expression control element operationally linked to the nucleic acid sequence encoding the Granzyme A polypeptide. The expression control elements are inserted in the viral vector to control and regulate the expression of the nucleic acid sequence. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons. Examples of expression control elements of use in these vectors includes, but is not limited to, lac system, operator and promoter regions of phage lambda, yeast promoters and promoters derived from polyoma, adenovirus, retrovirus or SV40. Additional operational elements include, but are not limited to, leader sequence, termination codons, polyadenylation signals and any other sequences necessary for the appropriate transcription and subsequent translation of the nucleic acid sequence encoding Granzyme A in the host system. The expression vector can contain additional elements necessary for the transfer and subsequent replication of the expression vector containing the nucleic acid sequence in the host system.

Examples of such elements include, but are not limited to, origins of replication and selectable markers. It will further be understood by one skilled in the art that such vectors are easily constructed using conventional methods (Ausubel et al., (1987) in “Current Protocols in Molecular Biology,” John Wiley and Sons, New York, N.Y.) and are commercially available.

Basic techniques for preparing recombinant DNA viruses containing a heterologous DNA sequence encoding the Granzyme A, are known in the art. Such techniques involve, for example, homologous recombination between the viral DNA sequences flanking the DNA sequence in a donor plasmid and homologous sequences present in the parental virus (Mackett et al., 1982, Proc. Natl. Acad. Sci. USA 79:7415-7419).

In certain embodiments, the method includes administering to the subject an effective amount of an APE1 Base excision repair inhibitor. In embodiments, the APE1 Base excision repair inhibitor comprises CRT0044876.

In certain embodiments, the method includes administering to the subject an effective amount of an APE1 Redox Inhibitor. In certain embodiments, the APE1 Redox Inhibitor comprises E3330.

Aspects of the disclosure further relate to compositions for treating and/or inhibiting cancer and kits containing such compositions. Compositions, such as therapeutic or pharmaceutical compositions, are provided that include a therapeutically effective amount of one or more of Granzyme A or a nucleic acid encoding the same plasmid, and HSP90 Inhibitor, and APE1 Base excision repair inhibitor, and/or APE1 Redox Inhibitor. It is desirable to prepare the pharmaceutical composition appropriate for the intended application, for example to inhibit or treat a cellular proliferative disorder. Accordingly, methods for making a medicament or pharmaceutical composition are included herein. Pharmaceutical compositions can be prepared for administration alone or with other active ingredients, such as other chemotherapeutics. When administered to a subject, the administration can be concurrent or sequential. Sequential administration can be separated by any amount of time, so long as the desired affect is achieved. Multiple administrations of the compositions described herein are also contemplated.

In embodiments, the composition includes an effective amount of an HSP90 inhibitor and an effective amount of Granzyme A. In certain embodiments, the HSP90 inhibitor is one or more of a small molecule inhibitor of HSP90 activity, an inhibitory RNA that inhibits the expression of HSP90 protein, or an antibody that specifically binds HSP90. In certain embodiments, the small molecule inhibitor of HSP90 activity is 17-AAG (Tanespimycin). In certain embodiments, an effective amount of Granzyme A, comprises administering to the subject a nucleic acid encoding Granzyme A, wherein the nucleic acid encoding Granzyme A is operatively linked to a promoter. In certain embodiments, the nucleic acid encoding Granzyme A is operatively linked to a promoter comprising an expression vector. In certain embodiments, the expression vector comprises pEF-Granzyme A plasmid DNA. In certain embodiments, the composition includes an effective amount of an APE1 Base excision repair inhibitor. In certain embodiments, the APE1 Base excision repair inhibitor comprises CRT0044876. In certain embodiments, the composition includes an effective amount of an APE1 Redox Inhibitor. In certain embodiments, the PE1 Redox Inhibitor comprises E3330.

Pharmaceutical compositions can be administered to subjects by a variety of routes. This includes oral, nasal (such as intranasal), ocular, buccal, enteral, intravitral, or other mucosal (such as rectal or vaginal) or topical administration. Alternatively, administration will be by orthotopic, intradermal subcutaneous, intramuscular, parentral intraperitoneal, or iv injection routes. Such pharmaceutical compositions are usually administered as pharmaceutically acceptable compositions that include physiologically acceptable carriers, buffers or other excipients.

Typically, preparation of a pharmaceutical composition (for example, for use as a medicament or in the manufacture of a medicament) entails preparing a pharmaceutical composition that is essentially free of pyrogens, as well as any other impurities that could be harmful to humans or animals. Inhibitors of HSP90 activity may be included in pharmaceutical compositions (including therapeutic and prophylactic formulations), which are typically combined together with one or more pharmaceutically acceptable vehicles or carriers and, optionally, other therapeutic ingredients.

To formulate the pharmaceutical compositions, the active ingredients can be combined with various pharmaceutically acceptable additives, as well as a base or vehicle for dispersion of the compound. Desired additives include, but are not limited to, pH control agents, such as arginine, sodium hydroxide, glycine, hydrochloric acid, citric acid, and the like. In addition, local anesthetics (for example, benzyl alcohol), isotonizing agents (for example, sodium chloride, mannitol, sorbitol), adsorption inhibitors (for example, Tween 80), solubility enhancing agents (for example, cyclodextrins and derivatives thereof), stabilizers (for example, serum albumin), and reducing agents (for example, glutathione) can be included. When the composition is a liquid, the tonicity of the formulation, as measured with reference to the tonicity of 0.9% (w/v) physiological saline solution taken as unity, is typically adjusted to a value at which no substantial, irreversible tissue damage will be induced at the site of administration. Generally, the tonicity of the solution is adjusted to a value of about 0.3 to about 3.0, such as about 0.5 to about 2.0, or about 0.8 to about 1.7.

Active ingredients can be dispersed in a base or vehicle, which can include a hydrophilic compound having a capacity to disperse the compound, and any desired additives. The base can be selected from a wide range of suitable compounds, including but not limited to, copolymers of polycarboxylic acids or salts thereof, carboxylic anhydrides (for example, maleic anhydride) with other monomers (for example, methyl (meth)acrylate, acrylic acid and the like), hydrophilic vinyl polymers, such as polyvinyl acetate, polyvinyl alcohol, polyvinylpyrrolidone, cellulose derivatives, such as hydroxymethylcellulose, hydroxypropylcellulose and the like, and natural polymers, such as chitosan, collagen, sodium alginate, gelatin, hyaluronic acid, and nontoxic metal salts thereof. Often, a biodegradable polymer is selected as a base or vehicle, for example, polylactic acid, poly(lactic acid-glycolic acid) copolymer, polyhydroxybutyric acid, poly (hydroxybutyric acid-glycolic acid) copolymer and mixtures thereof. Alternatively or additionally, synthetic fatty acid esters such as polyglycerin fatty acid esters, sucrose fatty acid esters and the like can be employed as vehicles. Hydrophilic polymers and other vehicles can be used alone or in combination, and enhanced structural integrity can be imparted to the vehicle by partial crystallization, ionic bonding, cross-linking and the like. The vehicle can be provided in a variety of forms, including fluid or viscous solutions, gels, pastes, powders, and microspheres.

Active ingredients can be combined with the base or vehicle according to a variety of methods, and release of the compound can be by diffusion, disintegration of the vehicle, or associated formation of water channels. In some circumstances, the compound is dispersed in microcapsules (microspheres) or nanocapsules (nanospheres) prepared from a suitable polymer, for example, isobutyl 2-cyanoacrylate (see, for example, Michael et al., J. Pharmacy Pharmacol. 43: 1-5, 1991), and dispersed in a biocompatible dispersing medium, which yields sustained delivery and biological activity over a protracted time.

Active ingredients can alternatively contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate. For solid compositions, conventional nontoxic pharmaceutically acceptable vehicles can be used which include, for example, pharmaceutical grades of mannitol, lactose, starch, magnesium stearate, sodium saccharin, talcum, cellulose, glucose, sucrose, magnesium carbonate, and the like.

Pharmaceutical compositions can also be formulated as a solution, microemulsion, or other ordered structure suitable for high concentration of active ingredients. The vehicle can be a solvent or dispersion medium containing, for example, water, ethanol, polyol (for example, glycerol, propylene glycol, liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols, such as mannitol and sorbitol, or sodium chloride in the composition. Prolonged absorption of the compound can be brought about by including in the composition an agent which delays absorption, for example, monostearate salts and gelatin.

Antibodies may be provided in lyophilized form and rehydrated with sterile water before administration, although they are also provided in sterile solutions of known concentration. In some examples, the antibody solution is then added to an infusion bag containing 0.9% sodium chloride, USP, and typically administered at a dosage of from 0.5 to 15 mg/kg of body weight. Considerable experience is available in the art in the administration of antibody drugs, which have been marketed in the U.S. since the approval of RITUXA® in 1997. Antibodies can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30 minute period if the previous dose was well tolerated.

For prophylactic and therapeutic purposes, the pharmaceutical compositions can be administered to the subject in a single bolus delivery, via continuous delivery (for example, continuous transdermal, mucosal or intravenous delivery) over an extended time period, or in a repeated administration protocol (for example, by an hourly, daily or weekly, repeated administration protocol). The therapeutically effective dosage of the compound can be provided as repeated doses within a prolonged prophylaxis or treatment regimen that will yield clinically significant results to alleviate one or more symptoms or detectable conditions associated with a targeted disease or condition as set forth herein.

Determination of effective dosages in this context is typically based on animal model studies followed up by human clinical trials and is guided by administration protocols that significantly reduce the occurrence or severity of targeted disease symptoms or conditions in the subject. Suitable models in this regard include, for example, murine, rat, porcine, feline, non-human primate, and other accepted animal model subjects known in the art. Alternatively, effective dosages can be determined using in vitro models (for example, immunologic and histopathologic assays). Using such models, only ordinary calculations and adjustments are required to determine an appropriate concentration and dose to administer a therapeutically effective amount of an active ingredient.

The appropriate dose will vary depending on the characteristics of the subject, for example, whether the subject is a human or non-human, the age, weight, and other health considerations pertaining to the condition or status of the subject, the mode, route of administration, and number of doses, time and route of administration, other drugs or treatments being administered concurrently, as well as the specific pharmacology of the therapeutic compositions for eliciting the desired activity or biological response in the subject. Dosage regimens can be adjusted to provide an optimum prophylactic or therapeutic response.

A therapeutically effective amount is also one in which any toxic or detrimental side effects of the compound and/or other biologically active agent is outweighed in clinical terms by therapeutically beneficial effects.

Therapeutic compositions can be delivered by way of a pump (see Langer, supra; Sefton, CRC Crit. Ref. Biomed. Eng. 14:201, 1987; Buchwald et al, Surgery 88:507, 1980; Saudek et al, N. Engl. J. Med. 321:574, 1989) or by continuous subcutaneous infusions, for example, using a mini-pump. An intravenous bag solution can also be employed. One factor in selecting an appropriate dose is the result obtained, as measured by the methods disclosed here, as are deemed appropriate by the practitioner. Other controlled release systems are discussed in Langer {Science 249: 1527-33, 1990).

In particular examples, therapeutic compositions are administered by sustained-release systems. Suitable examples of sustained-release systems include suitable polymeric materials (such as, semi-permeable polymer matrices in the form of shaped articles, for example films, or mirocapsules), suitable hydrophobic materials (for example as an emulsion in an acceptable oil) or ion exchange resins, and sparingly soluble derivatives (such as, for example, a sparingly soluble salt). Sustained-release compositions can be administered orally, parenterally, intracistemally, intraperitoneally, topically (as by powders, ointments, gels, drops or transdermal patch), or as an oral or nasal spray. Sustained-release matrices include polylactides (U.S. Pat. No. 3,773,919, EP 58,481), copolymers of L-glutamic acid and gamma-ethyl-L-glutamate (Sidman et al, Biopolymers 22:547-556, 1983, poly(2-hydroxyethyl methacrylate)); (Langer et al, J. Biomed. Mater. Res. 15:167-277, 1981; Langer, Chem. Tech. 12:98-105, 1982, ethylene vinyl acetate (Langer et al, Id.) or poly-D-(−)-3-hydroxybutyric acid (EP 133,988).

Polymers can be used for ion-controlled release. Various degradable and nondegradable polymeric matrices for use in controlled drug delivery are known in the art (Langer, Accounts Chem. Res. 26:537, 1993). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston et al, Pharm. Res. 9:425, 1992; and Pec, /. Parent. Sci. Tech. 44(2):58, 1990). Alternatively, hydroxyapatite has been used as a microcarrier for controlled release of proteins (Ijntema et al, Int. J. Pharm. 112:215, 1994). In yet another aspect, liposomes are used for controlled release as well as drug targeting of the lipid-capsulated drug (Betageri et al, Liposome Drug Delivery Systems, Technomic Publishing Co., Inc., Lancaster, Pa., 1993). Numerous additional systems for controlled delivery of therapeutic proteins are known (for example, U.S. Pat. Nos. 5,055,303; 5,188,837; 4,235,871; 4,501,728; 4,837,028; 4,957,735; and 5,019,369; 5,055,303; 5,514,670; 5,413,797; 5,268,164; 5,004,697; 4,902,505; 5,506,206; 5,271,961; 5,254,342; and 5,534,496).

The following examples are provided to illustrate particular features of certain embodiments. However, the particular features described below should not be construed as limitations on the scope of the disclosure, but rather as examples from which equivalents will be recognized by those of ordinary skill in the art.

### EXAMPLE

Materials and Methods

Cell Culture: UMUC3 human bladder cancer cells were obtained from the American Type Cell Culture (Manassas, Va.). Cells were cultured in DMEM media supplemented with 10% v/v fetal bovine serum (FBS) and 1% penicillin-streptomycin (10,000 units/mL) solution at 37° C. under carbon dioxide (5%) in an incubator. At approximately 80% confluence, cells were trypsinized using 0.25% trypsin-EDTA and seeded at a density of 10,000 cells/well in 96-well plates (Corning, Corning, N.Y., USA) for all transfection and cell viability experiments.

Synthesis of Polymers:

The synthesis and characterization of aminoglycoside-derived polymers (Paromomycin-GDE, Neomycin-GDE and Neomycin-RDE) were performed as follows. Briefly, sulphate-containing aminoglycosides neomycin and paromomycin were dissolved in nanopure water and passed through a Cl− ion exchange resin for 3 hr. The resulting sulphate-free aminoglycosides were reacted with GDE or RDE in a 1:2.2 molar ratio in a solvent mixture of water and N,N-dimethylformamide (DMF), (1.5:1 v/v) for 5 hr at 60° C. The reaction mixture was precipitated by using acetone, and was further purified by dialysis using a 3.5 kDa molecular weight cutoff (MWCO) dialysis membrane for 48 hr to remove unreacted aminoglycosides and diglycidylethers. The dialyzed material was lyophilized to obtain the purified polymers. Neomycin-GDE, Paromomycin-GDE, Neomycin-RDE polymers are abbreviated as NG, PG and NR respectively. These untargeted polymers and are also called parental polymers collectively in subsequent usage.

Construction of pEF Granzyme A Plasmid:

To construct the pEF-Granzyme A plasmid, full-length Granzyme A was amplified via PCR from the pET26B-Granzyme A plasma (Addgene plasmid #8823). Amplification was carried out using custom DNA oligonucleotides synthesized by Integrated DNA Technologies (Coralville, Iowa, USA) and using a Bio-Rad iCycler system with Q5 High Fidelity DNA Polymerase (New England Biolabs (NEB), Ipswich, Mass., USA) according to manufacturer protocols. The Amplified Granzyme A DNA was incubated with Proteinase K (NEB) according to manufacturer protocols, and subsequently purified using the Zymo Research DNA Clean & Concentrator MiniPrep (Zymo Research, Irvine, Calif., USA) according to manufacturer protocols. Purified Granzyme A DNA and pEF-GFP (purchased from Addgene, plasmid #11154), which contains the EGFP gene under the control of the EF1α promoter, were individually digested with Bam H1, Xho1 EcoRI-HF and Not1-HF (NEB) according to manufacturer protocols; this step removed EGFP from the pEF-GFP plasmid. Digested DNA fragments were gel-purified using the Zymoclean Gel DNA Recovery MiniPrep (Zymo Research). Purified digested Granzyme A and pEF-GFP DNA were ligated with T4 DNA Ligase (NEB) according to manufacturer protocols, resulting in the pEF-Granzyme A plasmid with the EF1α promoter driving expression of Granzyme A. Ligation reactions were transformed into chemically competent E. coli DH5α (NEB) and selected for on LB solid agar plates supplemented with 100 mg/L ampicillin. The resultant transformant pool was screened for correct clones using colony PCR, restriction digest mapping, and confirmed by DNA sequencing.

Granzyme Plasmid Cytotoxicity Study:

Cells were seeded at a density of 10,000 per well in a 96-well plate and allowed to attach for 18-24 hour. Different polymers (NR, PGC18, NGC18, JET PEI) were used to deliver the pEF-Granzyme plasmid at a weight ratio 25:1 polymer:pDNA with pEF-GFP used as control. Weight ratio of 25:1 polymer:pDNA was used in all cases. All transfections were carried out in presence of media containing 10% FBS and 75 ng pDNA was used per well. After 48 hour of incubation with the polyplexes the MTT ((3-(4,5-Dimethylthiazol-2-yl)-2,5-diphenyltetrazolium bromide)) assay was employed for determining the cell viability following treatment with different polymer:granzyme and polymer:gfp complexes (25:1) in UMUC3 cells. Untreated control wells were treated only with media (i.e., no polymer). Post 48 hour, 10 μL of the MTT reagent were added to the cells and they were incubated for 3 h at 37° C. following which, 50 μL dimethylsulfoxide:methanol (1:1) were added and incubated at room temperature for 30 min in a shaking incubator and absorbance at 570 nm was determined using a plate reader (Bio-Tek Synergy 2). The relative cell viability (%) was calculated from [ab]test/[ab]control×100%, where [ab]test and [ab]control are the absorbance values of the wells with polymers and control wells without polymers, respectively. For each sample, the final absorbance/cell viability was reported as the average of values measured from three wells in parallel.

HSP90 Inhibition and Granzyme a Synergy:

UMUC3 cells were seeded onto 96 well plate at a concentration of 10,000 cells per well and were allowed to attach for 24 hours. Based on previous studies PGC18 1:5 was chosen as the polymer to deliver the pDNA (pEF-Granzyme and pEF-GFP control). Before transfection the cells were pre-treated with HSP90 inhibitor 17-AAG at concentrations (1 uM, 3 uM and 5 uM) in DMEM media. All transfections were carried out in DMEM media containing 10% FBS and 75 ng of pDNA was used per well. MTT assay was performed after a period of 48 hours. 10 μL of the MTT reagent were added to the cells and they were incubated for 3 hours at 37° C. following which, 50 μL dimethylsulfoxide:methanol (1:1) were added and incubated at room temperature for 30 min in a shaking incubator and absorbance at 570 nm was determined using a plate reader (Bio-Tek Synergy 2). The relative cell viability (%) was calculated from [ab]test/[ab]control×100%, where [ab]test and [ab]control are the absorbance values of the wells with polymers and control wells without polymers, respectively. For each sample, the final absorbance/cell viability was reported as the average of values measured from three wells in parallel.

HSP90 siRNA and Granzyme A Transfection:

Similar to previous study 10,000 cells per well were seeded onto a 96 well plate. These cells were then transfected with PRC6 polymer containing HSP90 siRNA. These siRNA treated cells were then transfected with pGranzyme post 4 days of siRNA treatment. 2 days post pGranzyme transfection MTT assay was performed as mentioned previously.

Combination Treatment of HSP90 Inhibitor 17-AAG and pTRAIL:

UMUC3 cells were seeded onto 96 well plate at a concentration of 10,000 cells per well and were allowed to attach for 24 hours. Based on previous studies PGC18 1:5 was chosen as the polymer to deliver the pDNA (pEF-Trail and pEF-GFP control). Before transfection the cells were pre-treated with HSP90 inhibitor 17-AAG at concentrations (1 μM, 3 μM and 5 μM) in DMEM media. All transfections were carried out in DMEM media containing 10% FBS and 75 ng of pDNA was used per well. MTT assay was performed after a period of 48 hours. 10 μL of the MTT reagent were added to the cells and they were incubated for 3 h at 37° C. following which, 50 pt dimethylsulfoxide:methanol (1:1) were added and incubated at room temperature for 30 min in a shaking incubator and absorbance at 570 nm was determined using a plate reader (Bio-Tek Synergy 2). The relative cell viability (%) was calculated from [ab]test/[ab]control×100%, where [ab]test and [ab]control are the absorbance values of the wells with polymers and control wells without polymers, respectively. For each sample, the final absorbance/cell viability was reported as the average of values measured from three wells in parallel.

Reactive Oxygen Species Kinetics Assay:

Before performing transfections the plate of UMUC3 cells were stained using a DCFDA dye at a working concentration of 20 μM for 45 minutes at 37 degrees Celsius in the dark. The cells were then washed and treated with the relevant drug and pDNA combination. After a period of 48 hours ROS levels were measured using the Biotek Synergy 2 plate reader at an excitation wavelength of 485 nm and emission wavelength of 535 nm. Cells treated with 50 μM TBHP (Tert-Butyl hydrogel peroxide) were used as positive control. Blank readings were subtracted from all measurements and for each condition the fluorescence was reported as an average of the values measured from three wells in parallel.

JC-1 Assay for Mitochondrial Membrane Potential Analysis:

Using a seeding density of 10,000 cells per well UMUC3 cells were plated on to a 96 well plate and allowed to attach for 24 hours. Post 24 hours the media was removed and cells were stained using 5 μg/ml of JC-1 dye in media for 1 hour. The media containing the stain was removed and replaced with media based on different treatment conditions for 17-AAG and pGranzyme A transfections. Fluorescence was measured at emission wavelengths of 535 nm and 590 nm. This was done both 24 and 48 hours post transfection. For each sample the final value was presented as an average of the Ratio between 590 nm/535 nm measured from three wells in parallel.

Western Blotting for APE1 and NM23H1:

Expression of APE1 and NM23H1 in UMUC3 cells following delivery of pGranzyme A using polymer PGC18 (1:5) was determined using Western blots. UMUC3 cells were seeded at a density of 100,000 cells/well in a 6-well plate and treated with PGC18 (1:5) polymers complexed with 500 ng pGranzyme A plasmid/well or 500 ng pEF-GFP plasmid/well for 48 hr. These conditions were different from the transfection studies performed above to facilitate the collection of sufficient protein for Western blotting. Protein (30 μg) was separated on 7.5% or 12% MiniProtean Pre-cast gels (Biorad inc.) and transferred to nitrocellulose for 2 hour at 30 V. Membranes were blocked using 6% milk in PBS-Tween for a minimum of 1 hour prior to incubation with primary antibody (APE1, 1:1,000, EMD Millipore or NM23H1, 1:1000, Thermofisher Scientific) and anti-Actin, 1:4,000, Sigma A2066) overnight. Following three washes with PBS-Tween, membranes were incubated with HRP-conjugated secondary anti-rabbit or anti-mouse antibody (1:10,000 dilution, SantaCruz Biotechnology, Inc.) for 1.5 hours at room temperature in TBS-Tween. Membranes were washed three times in PBS-Tween followed by detection of the secondary conjugates with Supersignal Femto West (Thermofisher Scientific).

APE-1 Inhibitor and HSP90 Inhibitor Combination:

UMUC3 and A2780 cells were seeded onto 96 well plate at a concentration of 10,000 cells per well and were allowed to attach for 24 hours. Cell were treated with HSP90 inhibitor 17-AAG at concentrations (15 μM and 15 nM in UMUC-3 and A2780 cells respectively) and 1-15 μM of APE-1 inhibitor (CRT0044876 (BER) or E33330 (redox)) in DMEM media. All treatments were carried out in DMEM media containing 10% FBS and 1% PS. MTT assay was performed after a period of 72 hours. 10 μL of the MTT reagent were added to the cells and they were incubated for 3 hours at 37° C. following which, 50 μL dimethylsulfoxide:methanol (1:1) were added and incubated at room temperature for 30 min in a shaking incubator and absorbance at 570 nm was determined using a plate reader (Bio-Tek Synergy 2). The relative cell viability (%) was calculated from [ab]test/[ab]control×100%, where [ab]test and [ab]control are the absorbance values of the wells with polymers and control wells without polymers, respectively. For each sample, the final absorbance/cell viability was reported as the average of values measured from three wells in parallel.

HSP90 siRNA and APE-1 Inhibition:

For this experiment 10,000 A2780 ovarian cancer cells per well were seeded onto a 96 well plate. These cells were then transfected with PRC6 polymer containing HSP90 siRNA. These siRNA treated cells were then treated with CRT0044876 1.) Simultaneous treatment 2.) 2 days post transfection 3.) 4 days post transfection. Cell viability was assessed on 4th day for the simultaneous treatment and 2 day post transfection conditions and on the 6th day for 4 day post transfection treatment. 10 μL of the MTT reagent were added to the cells and they were incubated for 3 hours at 37° C. following which, 50 μL dimethylsulfoxide:methanol (1:1) were added and incubated at room temperature for 30 min in a shaking incubator and absorbance at 570 nm was determined using a plate reader (Bio-Tek Synergy 2). The relative cell viability (%) was calculated from [ab]test/[ab]control×100%, where [ab]test and [ab]control are the absorbance values of the wells with polymers and control wells without polymers, respectively. For each sample, the final absorbance/cell viability was reported as the average of values measured from three wells in parallel.

Statistical Analysis:

All experiments were performed in triplicates and the results are expressed as mean±one standard deviation using Sigmaplot 13.0 (Systat software, Inc.). Student's t-Test was used to assess statistical significance of difference between group means. p-values <0.05 with respect to corresponding controls are considered statistically significant

Results

pGranzyme a Mediated Cancer Cell Ablation:

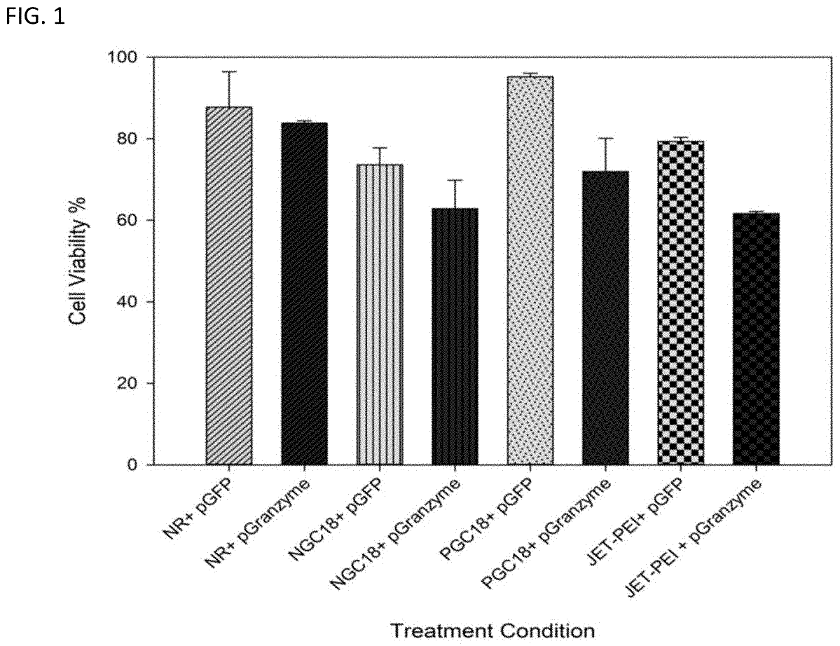

Granzyme A protein is released by Cytotoxic T-cells and Natural Killer cells to induce programmed cell death. Granzyme A is unique in its mode of cell death and does not follow the Caspase mediated apoptotis pathway used by the equally abundant Granzyme B. Polymers derived from the Aminoglycosides were employed to deliver a newly constructed Granzyme A protein producing plasmid. A pEF-GFP plasmid was employed as a control for these experiments. Cancer cell death was measured after 48 hrs using MTT assay. PGC18 (1:5) and JET-PEI complexed with pGranzyme A showed a modest but statistically significant increase in cell death compared to the pEF-GFP control (see FIG. 1). Around 25-30% cell death was observed in these conditions

Interaction of pGranzyme A with PARP Inhibitor (Olaparib):

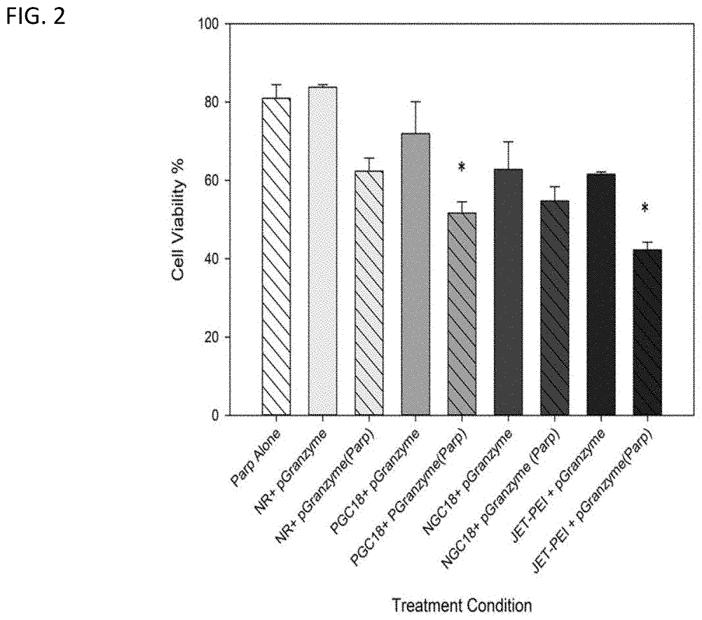

A PARP inhibitor was used to inhibit PARP controlled DNA repair activity and thereby increase the cancer cell ablation induced by pGranzyme A. For this purpose the cells were treated with PARP inhibitor Olaparib at a concentration of 2 μM before delivery of pGranzyme A plasmid. Significant increase in cell death was again observed in PGC18 (1:5) and JET-PEI complexed with pGranzyme A plasmids in the presence of PARP (see FIG. 2). This increase in cancer ablation was additive rather than synergistic.

Synergistic Ablation Effect of HSP90 Inhibitor (17-AAG) and pGranzyme:

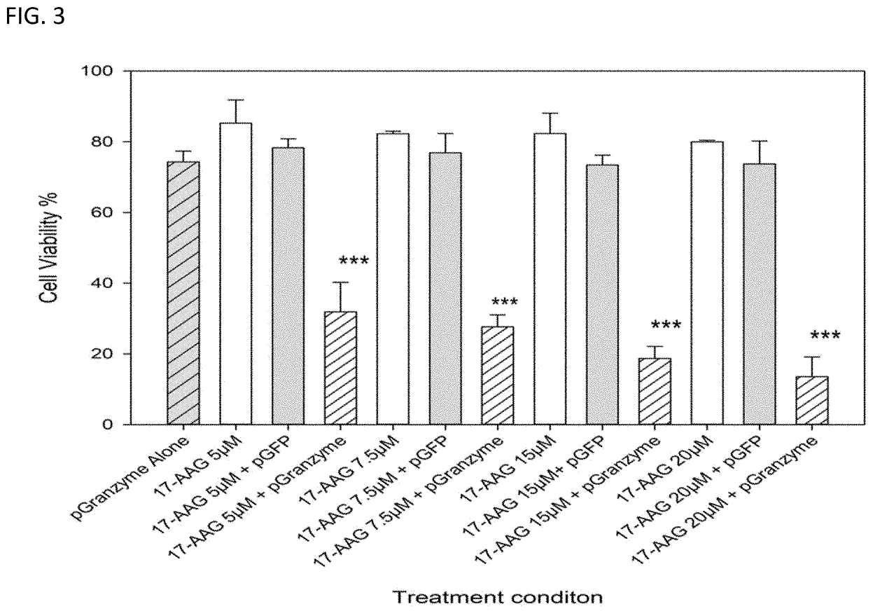

Heat shock proteins 60, 70 and 90 have been shown to transport Granzyme A into the mitochondria. Among these heat shock proteins, HSP90 has been observed to not be important for granzyme A transport and has a mitochondria protective function. Cells were treated with HSP90 inhibitor (17-AAG) before performing the transfection using PGC18 (1:5) complexed with pGranzyme A. The plasmid pEF-GFP was employed as a control for these experiments. Significant reduction in cancer cell viability was observed in the pGranzyme A and 17-AAG combination with maximum cell death (85-90%) observed when using 20 μM of 17-AAG in combination with pGranzyme A (see FIG. 3).

HSP90 siRNA Combination with pGranzyme A:

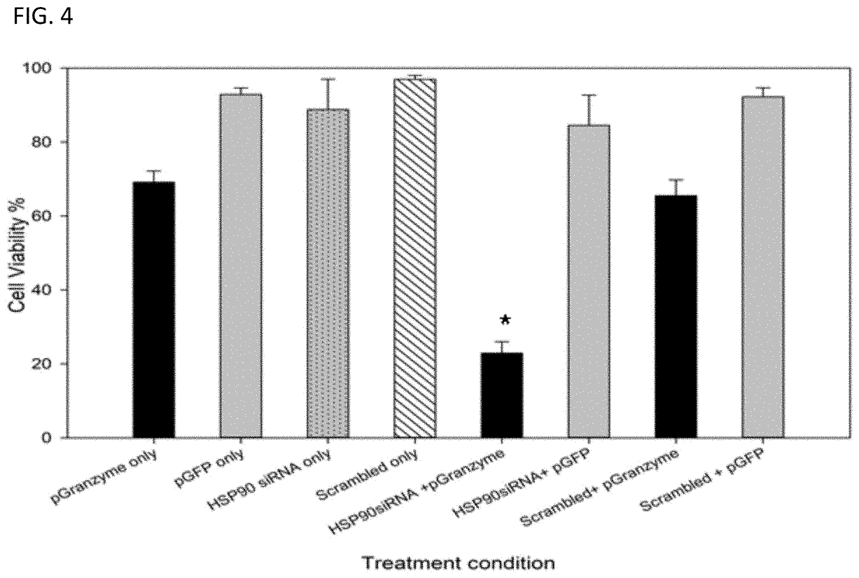

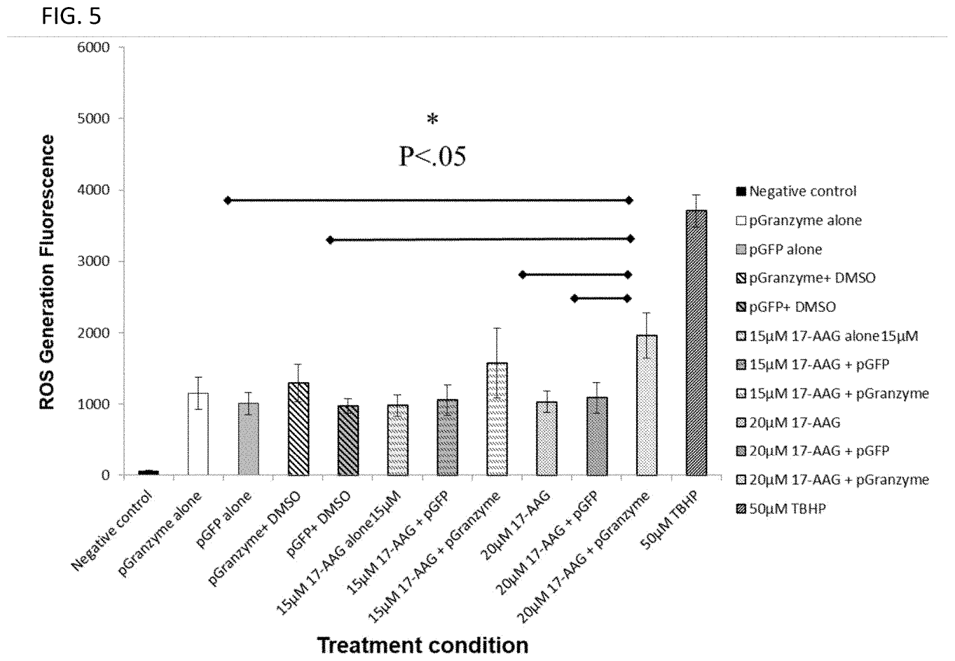

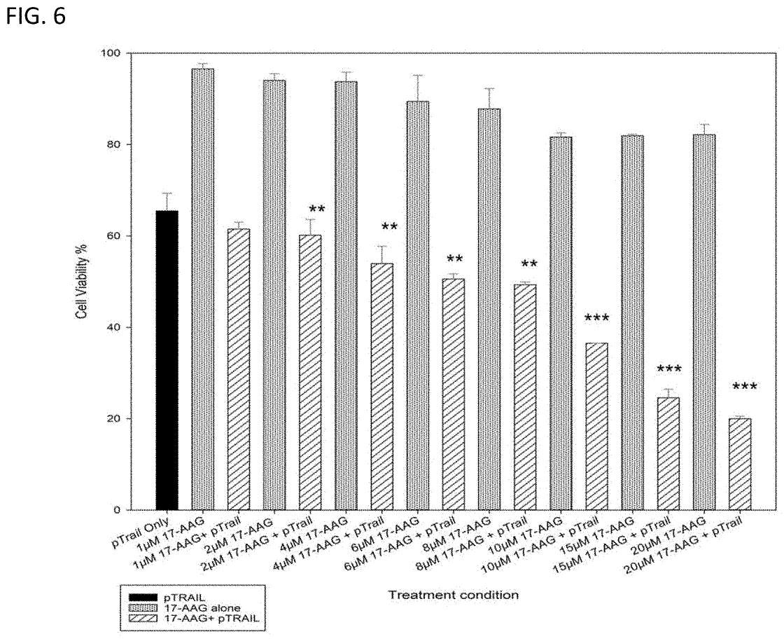

It was hypothesized that a similar ablation effect can be achieved by knocking down HSP90 using siRNA. The UMUC3 cells were transfected with either HSP90 siRNA or Scrambled siRNA complexed in PRC6 polymer. 96 hrs after siRNA treatment the cells were transfected with PGC18 polymers complexed with pGranzyme A or pEF-GFP. Cancer cell death induced was then measured after 48 hrs and around 75% cell death was observed. This provided a secondary confirmation of the synergistic effect of pGranzyme A (see FIG. 4). As shown in FIG. 5, generation of ROS in UMUC3 cells in different treatment conditions was measured after 36 h using transformation of 2′,7′-dichlorofluorescin diacetate (DCFDA) to the fluorescent 2′,7-dichlorofluorescein (DCF compound). Data are represented as the mean mean±one standard deviation of three independent experiments. Statistical Analysis was performed using one-way ANOVA. As shown in FIG. 6, cancer cell ablation efficiency of pTRAIL in the presence of HSP90 inhibitor 17-AAG. Transfection performed using PGC18 Polymer:pTRAIL complexes (25:1) in UMUC3 cells. Data are presented as mean±one standard deviation of three independent experiments (n=3). * indicates p-value <0.05 comparing treatment with pTRAIL polyplexes in presence of 17-AAG to pTRAIL polyplex only treatment. Statistical Analysis performed using student's t-Test.

Ablation of Ovarian Cancer Cell Line A2780 by Synergistic Effect of HSP90 Inhibitor 17-AAG and pGranzyme A:

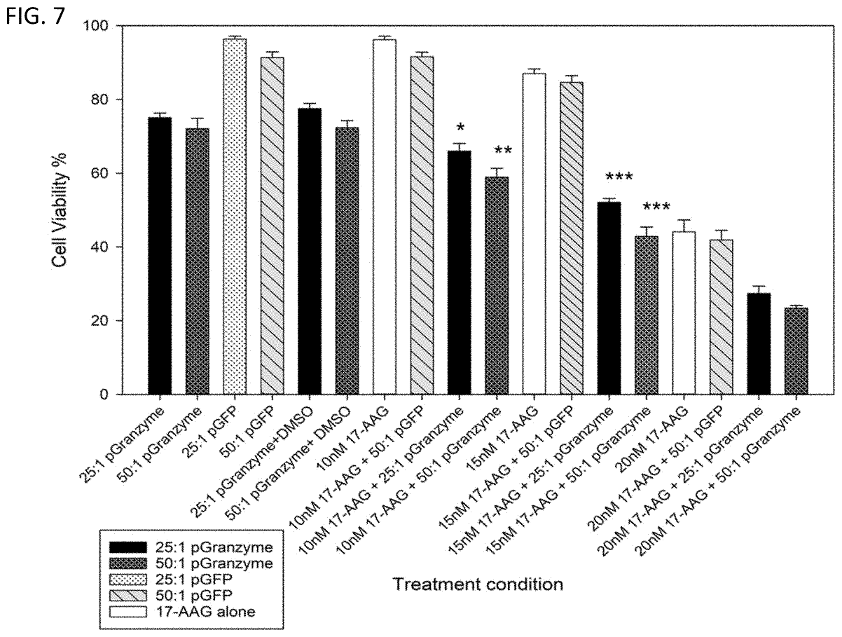

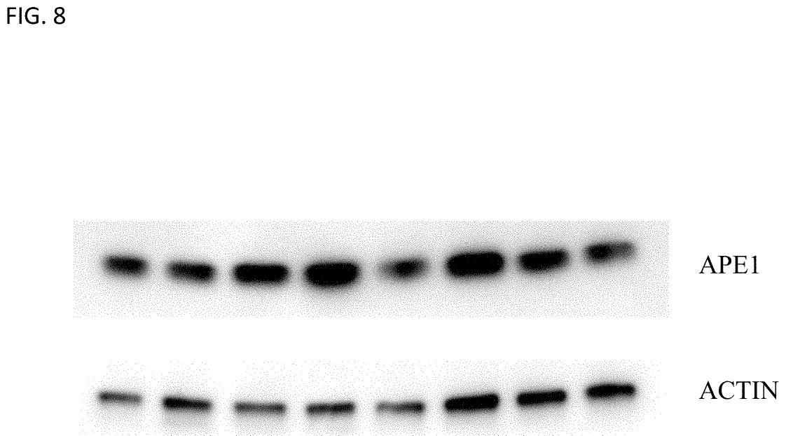

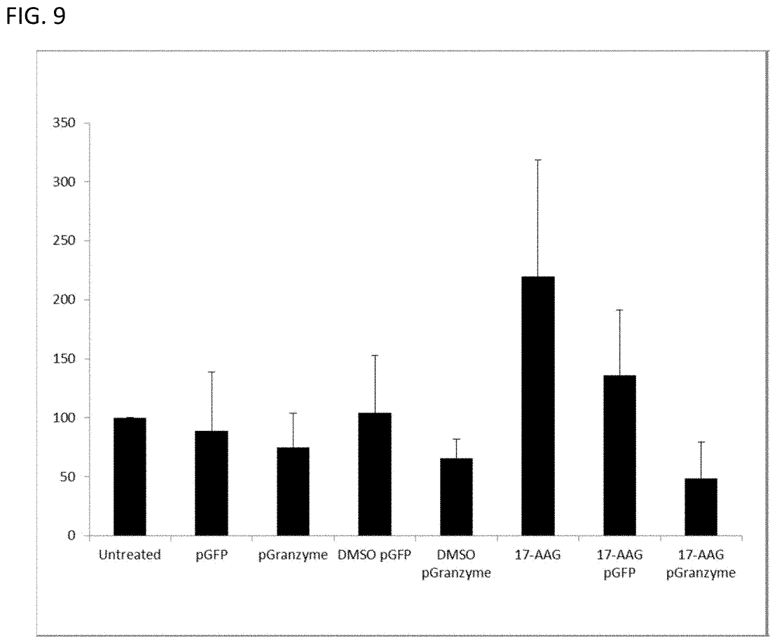

The effect of the HSP90 inhibitor and pGranzyme A combination treatment were studied in Ovarian cancer cell line A2780. For this experiment the polymer to pDNA ratios of 25:1 and 50:1 were used. The cells were transfected with PGC18 (1:5) polymers complexed with either pGranzyme A or pEF-GFP. Three different concentrations of 17-AAG (10 nM, 15 nM and 20 nM) were used and cell viability was measured after 48 hours. Significant decrease in cell viability was observed in 10 nM and 15 nM 17-AAG treated conditions. 20 nM of the 17-AAG drug was found to be toxic to the cells (see FIGS. 7-9).

Cancer Cell Ablation Using 17-AAG and APE-1 Inhibitor (CRT0044876) Combination:

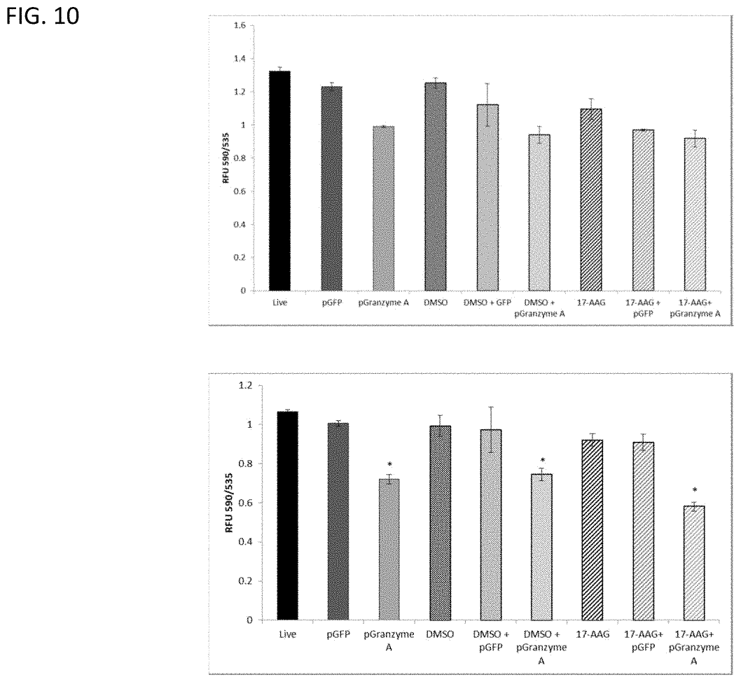

Ovarian cancer cell line (A2780) and Bladder cancer cell line (UMUC3) were treated with multiple doses of APE-1 Inhibitor combined with 15 nM of 17-AAG in A2780 cells or 15 μM of 17-AAG in UMUC3 cells. Significant decrease in cancer cell viability was observed in both A2780 and UMUC3 cells (see FIG. 10).

Cancer Cell Ablation Using APE-1 Redox Inhibitor (E3330) and 17-AAG Combination:

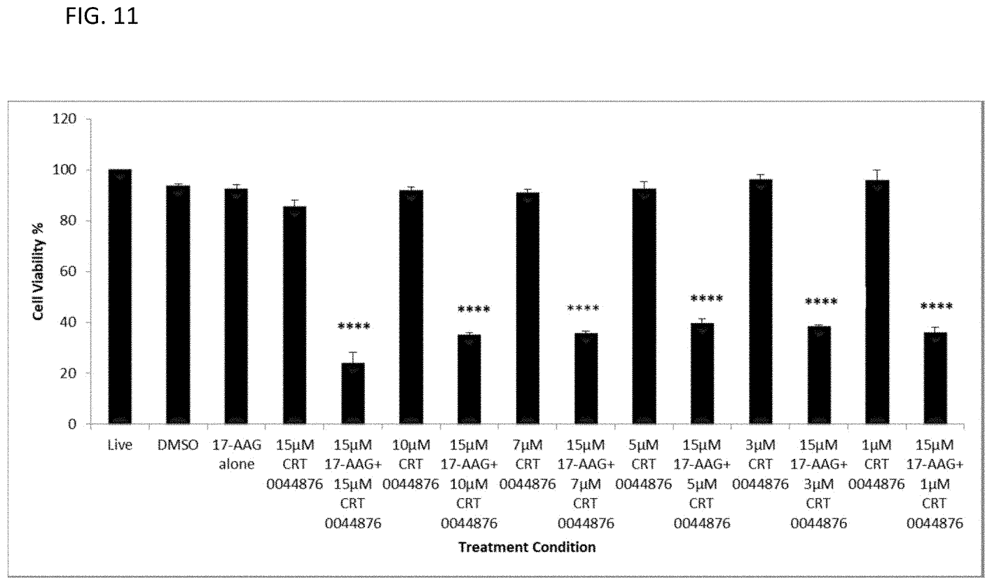

Ovarian cancer cell line A2780 was treated with a combination of HSP90 Inhibitor 17-AAG and APE-1 Redox pathway inhibitor E3330. A single dose of 15 nM 17-AAG was used for all cells and Different doses of E3330 ranging from 1 μM to 15 μM was used. Significant decrease in cell viability was observed in the combinations (see FIG. 11).

HSP90 siRNA and APE-1 Inhibitor (CRT0044876) Combination Treatment in A2780 Ovarian Cancer Cell Line:

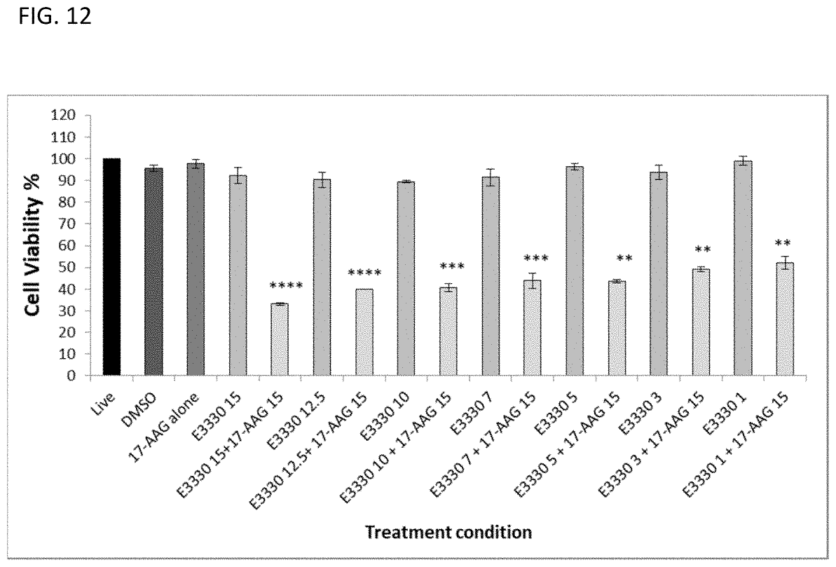

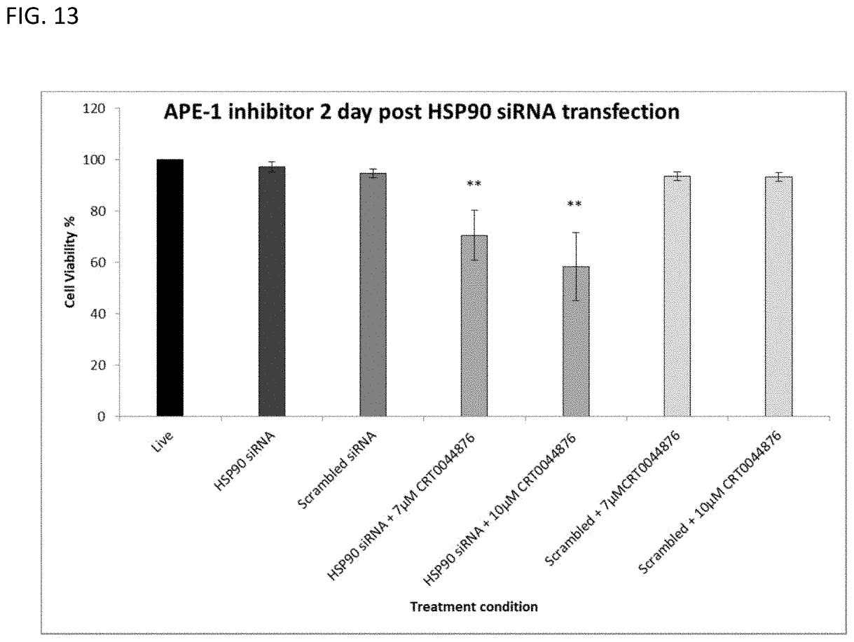

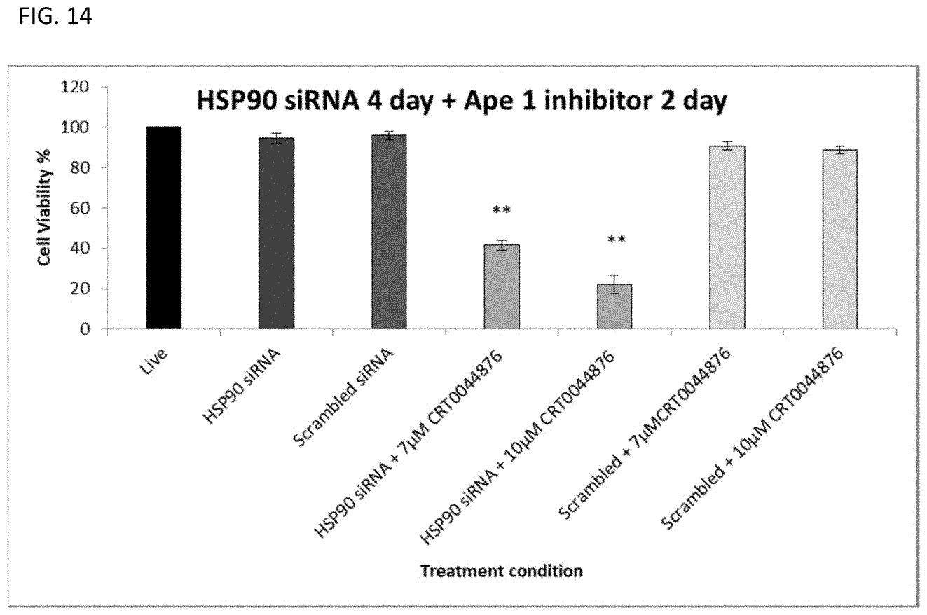

Three treatment conditions were used for treating the A2780 cell line. 1.) A simultaneous treatment of HSP90 siRNA transfection and APE-1 inhibitor for 4 days. 2.) APE-1 Inhibitor added 2 days post HSP90 siRNA transfection 3.) APE-1 inhibitor added after 4 days of siRNA treatment. Very minimal/no cell death was observed with simultaneous treatment. Treating cells after 4 days siRNA treatment showed an increase in cell death with the combination treatment. Very minimal cell death was observed in the case of all scrambled siRNA treatments (see FIG. 12-14).

ROS Generation with HSP90 Inhibitor and APE-1 Inhibitor (CRT0044876) Combination:

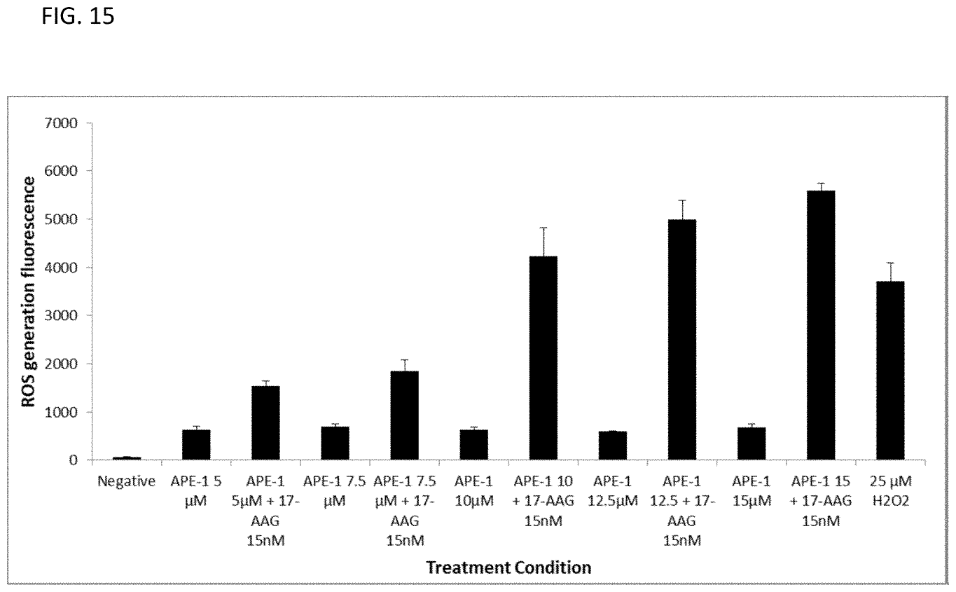

As shown in FIG. 15, generation of ROS in A2780 cells in different combination treatment conditions was measured after 36 hours using transformation of 2′,7′-dichlorofluorescin diacetate (DCFDA) to the fluorescent 2′,7-dichlorofluorescein (DCF compound). Data are represented as the mean mean±one standard deviation of three independent experiments. Statistical Analysis was performed using one-way ANOVA.

### REFERENCES

Chowdhury, D., & Lieberman, J. (2008). Death by a thousand cuts: granzyme pathways of programmed cell death. Annu. Rev. Immunol., 26, 389-420.
Lieberman, J. (2010). Granzyme A activates another way to die. Immunological reviews, 235(1), 93-104.
Hayes, M. P., Berrebi, G. A., & Henkart, P. A. (1989). Induction of target cell DNA release by the cytotoxic T lymphocyte granule protease granzyme A. Journal of Experimental Medicine, 170(3), 933-946.
Beresford, P. J., Xia, Z., Greenberg, A. H., & Lieberman, J. (1999). Granzyme A loading induces rapid cytolysis and a novel form of DNA damage independently of caspase activation. Immunity, 10(5), 585-595.
Beresford, P. J., Zhang, D., Oh, D. Y., Fan, Z., Greer, E. L., Russo, M. L., . . . & Lieberman, J. (2001). Granzyme A activates an endoplasmic reticulum-associated caspase-independent nuclease to induce single-stranded DNA nicks. Journal of Biological Chemistry, 276(46), 43285-43293.
Martinvalet, D., Zhu, P., & Lieberman, J. (2005). Granzyme A induces caspase-independent mitochondrial damage, a required first step for apoptosis. Immunity, 22(3), 355-370.
Shi, L. I. A. N. F. A., Kraut, R. P., Aebersold, R., & Greenberg, A. H. (1992). A natural killer cell granule protein that induces DNA fragmentation and apoptosis. Journal of Experimental Medicine, 175(2), 553-566.
Sutton, V. R., Vaux, D. L., & Trapani, J. A. (1997). Bcl-2 prevents apoptosis induced by perforin and granzyme B, but not that mediated by whole cytotoxic lymphocytes. The Journal of Immunology, 158(12), 5783-5790.
Fan, Z., Beresford, P. J., Oh, D. Y., Zhang, D., & Lieberman, J. (2003). Tumor suppressor NM23-H1 is a granzyme A-activated DNase during CTL-mediated apoptosis, and the nucleosome assembly protein SET is its inhibitor. Cell, 112(5), 659-672.
Pardo, J., Bosque, A., Brehm, R., Wallich, R., Naval, J., Müllbacher, A., & Simon, M. M. (2004). Apoptotic pathways are selectively activated by granzyme A and/or granzyme B in CTL-mediated target cell lysis. J Cell Biol, 167(3), 457-468.
Chowdhury, D., Beresford, P. J., Zhu, P., Zhang, D., Sung, J. S., Demple, B. & Lieberman, J. (2006). The exonuclease TREX1 is in the SET complex and acts in concert with NM23-H1 to degrade DNA during granzyme A-mediated cell death. Molecular cell, 23(1), 133-142.
Fan, Z., Beresford, P. J., Zhang, D., Xu, Z., Novina, C. D., Yoshida, A., & Lieberman, J. (2003). Cleaving the oxidative repair protein Ape1 enhances cell death mediated by granzyme A. Nature immunology, 4(2), 145-153.
Zhu, P., Zhang, D., Chowdhury, D., Martinvalet, D., Keefe, D., Shi, L., & Lieberman, J. (2006). Granzyme A, which causes single-stranded DNA damage, targets the double-strand break repair protein Ku70. EMBO reports, 7(4), 431-437.
Usmani, S. Z., Bona, R., & Li, Z. (2009). 17 AAG for HSP90 Inhibition in Cancer-From Bench to Bedside. Current molecular medicine, 9(5), 654-664.
Neckers, L., Kern, A., & Tsutsumi, S. (2007). Hsp90 inhibitors disrupt mitochondrial homeostasis in cancer cells. Chemistry & biology, 14(11), 1204-1206.
Karkoulis, P. K., Stravopodis, D. J., Margaritis, L. H., & Voutsinas, G. E. (2010). 17-Allylamino-17-demethoxygeldanamycin induces downregulation of critical Hsp90 protein clients and results in cell cycle arrest and apoptosis of human urinary bladder cancer cells. BMC cancer, 10(1), 481.

While this disclosure has been described with an emphasis upon particular embodiments, it will be obvious to those of ordinary skill in the art that variations of the particular embodiments may be used, and it is intended that the disclosure may be practiced otherwise than as specifically described herein. Features, characteristics, compounds, or examples described in conjunction with a particular aspect, embodiment, or example of the invention are to be understood to be applicable to any other aspect, embodiment, or example of the invention. Accordingly, this disclosure includes all modifications encompassed within the spirit and scope of the disclosure as defined by the following claims. We therefore claim as our invention all that comes within the scope and spirit of these claims.

We claim:

1. A method for inhibiting and/or treating cancer in a subject in need thereof, comprising:
administering to a subject an effective amount of an HSP90 inhibitor; and
administering to the subject an effective amount of Granzyme A, thereby treating and/or inhibiting the cancer in the subject in need thereof.

2. The method of claim 1, further comprising selecting a subject with cancer or suspected of having cancer.

3. The method of claim 1, wherein the HSP90 inhibitor is one or more of a small molecule inhibitor of HSP90 activity, an inhibitory RNA that inhibits the expression of HSP90 protein, or an antibody that specifically binds HSP90.

4. The method of claim 3, wherein the small molecule inhibitor of HSP90 activity is 17-AAG (Tanespimycin).

5. The method of claim 1, wherein administering to the subject an effective amount of Granzyme A, comprises administering to the subject a nucleic acid encoding Granzyme A, wherein the nucleic acid encoding Granzyme A is operatively linked to a promoter.

6. The method of claim 5, wherein the nucleic acid encoding Granzyme A is operatively linked to a promoter comprises an expression vector.

7. The method of claim 6, were the expression vector comprises pEF-Granzyme A plasmid DNA.

8. The method of claim 1, further comprising:
administering to the subject an effective amount of an APE1 Base excision repair inhibitor.

9. The method of claim 8, wherein the APE1 Base excision repair inhibitor comprises CRT0044876.

10. The method of claim 1, further comprising:
administering to the subject an effective amount of an APE1 Redox Inhibitor.

11. The method of claim 10, wherein the APE1 Redox Inhibitor comprises E3330.

12. A composition, comprising:
an effective amount of an HSP90 inhibitor; and
an effective amount of Granzyme A.

13. The composition of claim 12, wherein the HSP90 inhibitor is one or more of a small molecule inhibitor of HSP90 activity, an inhibitory RNA that inhibits the expression of HSP90 protein, or an antibody that specifically binds HSP90.

14. The composition of claim 13, wherein the small molecule inhibitor of HSP90 activity is 17-AAG (Tanespimycin).

15. The composition of claim 12, wherein administering to the subject an effective amount of Granzyme A, comprises administering to the subject a nucleic acid encoding Granzyme A, wherein the nucleic acid encoding Granzyme A is operatively linked to a promoter.

16. The composition of claim 15, wherein the nucleic acid encoding Granzyme A is operatively linked to a promoter comprises an expression vector.

17. The composition of claim 16, were the expression vector comprises pEF-Granzyme A plasmid DNA.

18. The composition of claim 12, further comprising an effective amount of an APE1 Base excision repair inhibitor.

19. The composition of claim 18, wherein the APE1 Base excision repair inhibitor comprises CRT0044876.

20. The composition of claim 12, further comprising an effective amount of an APE1 Redox Inhibitor, such as E3330.
